(19) United States
(12) Patent Application Publication
(10) Pub. No.: US 2023/0132897 A1
(43) Pub. Date: May 4, 2023

(54) PAD COMPRISING A PRESSURE ELEMENT

(71) Applicant: BAUERFEIND AG, Zeulenroda-Triebes (DE)

(72) Inventors: André BECK, Zeulenroda-Triebes (DE); Hans B. BAUERFEIND, Zeulenroda-Triebes (DE)

(73) Assignee: BAUERFEIND AG, Zeulenroda-Triebes (DE)

(21) Appl. No.: 17/914,804

(22) PCT Filed: Mar. 24, 2021
(86) PCT No.: PCT/EP2021/057547

§ 371 Date: Sep. 27, 2022

(30) Foreign Application Priority Data
Mar. 27, 2020 (DE) 10 2020 203 999.2

(51) Int. Cl. A61F 5/30

(52) U.S. Cl. CPC A61F 5/30

(57) ABSTRACT

A pad, in particular for orthopedic aids, comprising a pad pressure element. The invention also relates to orthoses, bandages, or compression garments which comprise pads according to the invention.

The invention relates to pads, in particular for orthopedic aids and sports aids, comprising a pad pressure element. The invention also relates to the use of the pads according to the invention in orthoses, bandages, or compression garments, as well as to orthoses, bandages, or compression garments which comprise the pads according to the invention.

Pads are known in various designs and for various prophylactic and therapeutic applications. Various pads are known, for example, from DE 27 22 563 C2, EP 0 598 291 A1, EP 1 688 107 A1 and EP 0 600 218 A2.

Pads consisting of two materials or components are also known from DE 297 01 001 U1. EP 0 496 071 A1 describes a pressure pad consisting of a softer material, in which at least one friction core consisting of a hard or incompressible material is arranged. Said friction core is intended to be used for a friction massage, i.e., a massage by means of dynamic friction of pain points, which is produced by the movement of the core relative to the soft tissue of the pad wearer.

The pads from the prior art can exert a pressure on the adjacent tissue over a wide area or can distribute and/or shield pressure that arises on the surface. However, these pads do not allow or do not sufficiently allow for application of a specific pressure, for example a stronger or weaker pressure to specific points.

The pressure also acts perpendicularly to the skin in pads from the prior art. A lateral pressure, which in particular leads to a movement of the pressure elements and thus brings about a massaging effect beyond a simple pressure, is therefore not possible.

The technical problem addressed by the present invention is therefore to provide improved pads, in particular pads of the kind which, in addition to an areal pressure, can exert a specific punctiform pressure. In particular, the technical problem addressed by the present invention is to provide a pad which results in an improved massaging effect, in particular a massaging effect by means of the movement of the pressure body or the muscle.

The technical problem addressed by the invention is solved by providing a pad according to claim 1.

The technical problem addressed by the invention is solved by providing a pad for orthopedic aids, comprising a pad base body consisting of a first material and at least one pad pressure element consisting of a second material, the pad pressure element being embedded in the pad base body at least in part, and the first material of the pad base body being softer than the second material of the pad pressure element, the pad pressure element comprising at least two rows of at least three elevations each, the at least two rows of elevations not extending in parallel at least in one portion and the elevations protruding from a base element of the pad pressure element.

Surprisingly, it was found that elevations, for example studs or knobs, which are arranged in rows, can thus act in a targeted manner on muscle, since they follow the course of the muscle fiber, and that this is particularly effective when there are at least two rows, i.e., multiple rows of elevations, which follow the individual fascicles of a muscle.

Skeletal muscle consists of several fascicles that converge at the tendon-bone insertion and are connected to the bone there. As seen from the tendon-bone insertion, the muscle fibers usually only diverge from one another at a small angle so as to spread out and then further extend approximately in parallel.

The region of the at least two rows of elevations in the pad which does not extend in parallel serves to contact the muscle fiber region in the vicinity of the tendon-bone insertion, in which the muscle fibers also do not extend in parallel, but rather diverge. However, the rows of elevations may also extend in parallel with one another in regions, in particular when the rows are extended, such that the elevations in the regions extending in parallel press on the muscle fibers extending approximately in parallel.

In the context of the present invention, a pad is in particular understood to mean a pressure pad, the shape of which is formed in particular by the pad base body. The pad base body may have any suitable shape. Conventional pads typically have planar bases. Projections can then protrude from a base of this kind. A pad according to the invention in particular comprises a first base or base surface which, when the pad is being used, faces the user. A pad is preferably designed to be substantially planar. A pad of this kind then preferably comprises a second base or base surface which, when the pad is being used, faces away from the user. The stud-shaped elevations point from the pad base body in the direction of the user.

The typical use of a pad is known to a person skilled in the art. Pads, and also the pads according to the invention, are used in particular to exert a pressure on particular body parts, for example in the region of the back or in the region of the kneecap. The pads are positioned and pressed onto the corresponding body region by means of additional devices, in particular bandages or orthoses.

The elevations, in particular studs or knobs, of the at least two rows are preferably designed to be rigid. This has the advantage that the movement of the joints adjoining the muscle causes a shortening or stretching of the muscle. In the case of a rigid design for the elevations, the muscle moves away under the elevations and thus advantageously massages itself.

In a preferred embodiment, the pad pressure element comprises three to six rows of at least three elevations each.

In a preferred embodiment, the pad pressure element comprises three or four rows of at least four elevations each.

In a preferred embodiment, the rows comprise at least four elevations. A person skilled in the art can easily determine the suitable number and spacing of the elevations for the intended use themselves.

In a preferred embodiment, the rows each comprise four to twelve elevations.

In a preferred embodiment, the rows each comprise four to ten elevations.

In a preferred embodiment, the rows each comprise five to nine elevations.

In a preferred embodiment, two adjacent rows of the at least two rows of at least three elevations each extend at an angle of at least 3° in the non-parallel portion. In a preferred embodiment, the at least two rows of at least three elevations each in the pad base body extend at an angle of at least 3°.

In a preferred embodiment, two adjacent rows of the at least two rows of at least three elevations each extend at an angle of at least 3° and at most 25° to one another in the non-parallel portion.

A person skilled in the art can readily determine the suitable angle of the adjacent rows, since said person knows the course of individual muscle fibers of different muscle groups.

In a preferred embodiment, the at least two rows of at least three elevations each in the pad base body are positioned in such a way that they are located in the region of muscle strands when the pad is applied and can exert pressure on said muscle strands.

When manufacturing the pad, a person skilled in the art can determine the suitable positioning and dimensioning of the pad, pad pressure element and elevations in the pad base body depending on the intended use, i.e., depending on the muscle on the muscle fiber strands of which pressure is to be exerted by means of the elevations.

In a preferred embodiment, the pad pressure element comprises at least one hole, at least two elevations on the pad pressure element being positioned at the edge of the at least one hole.

In a preferred embodiment, the at least two elevations are connected to one another via the web spanning the hole.

In a preferred embodiment, at least two pairs of elevations are positioned at the edge of the hole and are each connected to one another via a web spanning the hole, the webs of the two pairs of elevations intersecting, preferably approximately in the center of the hole.

In a preferred embodiment, the hole with the two pairs of elevations at the edge and the intersecting webs forms a spoke cross. The hole and preferably the spoke cross are preferably positioned in the pad in such a way that, when the pad is applied, they lie on the tendon-bone insertion. On account of the spoke cross design, the elevations are mounted so as to be movable, in particular more movable than the elevations of the at least two rows. Therefore, the elevations positioned at the hole can advantageously move toward one another or away from one another directly at the tendon-bone insertion, in particular at their ends that are remote from the base element, when the pad is being pressed perpendicularly onto the patient's skin. The movement of the elevations toward or away from one another advantageously constitutes a lateral movement which is caused by the perpendicular pressure of the pad on the skin and which leads to an improved massage. It was advantageously found that this lateral movement takes place chaotically, which increases the massaging effect.

In comparison with the elevations of the rows, the elevations additionally mounted in the manner of spokes are substantially more freely and movably resilient, and thus in particular also more chaotic. This effect is advantageous, since it is more difficult for the mechanoreceptors lying directly under the surface of the skin to adjust to the resilient movement of the elevations, which repeatedly changes somewhat. This leads to an extended effect of the massage.

While a movement of the elevations away from one another leads to a laterally directed pulling movement, the movement toward one another leads to a laterally directed pushing movement. Surprisingly, it has been found that this movement is also clearly transmitted to the skin when the stud-shaped elements are embedded at least in part, for example molded, in the softer pad base body.

Therefore, in this preferred embodiment, the elevations of the at least two rows cause a pressure effect on the muscle fibers of a muscle, which results in a massaging effect by means of the movement of the muscle and, at the same time, causes a massaging effect on the tendon-bone insertion by means of the movement of the elevations at the hole. Therefore, two different massaging options are combined on two different points of attack, rigid elevations acting on moving muscle fibers in the first massaging unit and moving elevations acting on the rigid tendon-bone insertion in the second massaging unit.

In a preferred embodiment, another elevation is located on and/or next to the point of intersection of the two webs.

In a preferred embodiment, the elevations positioned at the edge of the hole are assigned to the at least two rows of at least three elevations each.

In a preferred embodiment, the hole is positioned in the end region of the at least two rows of at least three elevations each.

In a preferred embodiment, the hole in the pad base body is positioned in such a way that the elevations positioned at the edge of the hole are located in the region of a tendon-bone insertion when the pad is applied and can exert pressure on said tendon-bone insertion.

Preferably, therefore, the elevations of the rows and the hole or rather the elevation of the hole are positioned relative to one another in the pad in such a way that the elevations of the rows act on the muscle fibers and follow the course of the muscle fibers, while the hole and the elevations assigned to the hole act on the tendon-bone insertion. If the rows with elevations begin in the region of the hole, the elevations in the rows act accordingly on the muscle fiber region which lies close to the tendon-bone insertion, i.e., on the end region of a muscle. Of course, the rows may be extended such that they also act on regions of the muscle fibers that are more distant from the tendon-bone insertion.

Of course, it is also possible for a plurality of units of rows extending one next to the other to be provided, such that individual units can act on different courses of muscle fibers. For example, in the case of an elbow pad, it is possible for the hole with the assigned elevations to be in the region of the elbow and for at least two rows of elevations to extend in the direction of the forearm, i.e., act on muscle fibers of the forearm, while at least two further rows of elevations are positioned in the direction of the upper arm, i.e., act on muscle fibers of the upper arm. The different units of rows of elevations may also be positioned so as to be movable relative to one another.

In a preferred embodiment, the pad pressure element comprises at least two additional rows of at least three elevations each, the at least two additional rows of elevations extending so as to be offset by approximately 180° with respect to the first at least two rows of elevations.

The at least two rows of elevations according to the invention are thus preferably mirrored, particularly preferably mirrored in the region of the preferred hole.

In a preferred embodiment, the pad pressure element comprises at least two additional rows of at least three elevations each, the at least two additional rows of elevations extending so as to be offset in an angular range of from 75° to 105° and/or so as to be offset in an angular range of from 165° to 205° with respect to the first at least two rows of elevations.

In a preferred embodiment, the pad pressure element comprises a first unit of at least two rows of at least three elevations each, a second unit of at least two rows of at least three elevations each, and a third unit of at least two rows of at least three elevations each, the at least two rows of the first unit and the at least two rows of the second unit being offset in an angular range of from 75° to 105°, and the at least two rows of the second unit and the at least two rows of the third unit being offset in an angular range of from 75° to 105°. Particularly preferably, the two rows of the first unit and the two rows of the third unit are offset in an angular range of from 165° to 205° with respect to one another.

A pad of this kind having two or three units of rows of elevations which are offset at a specific angle with respect to one another can advantageously be used, for example, as a shoulder pad. In this case, one unit of rows may extend in the direction of the chest, one unit of rows may extend in the direction of the back, and one unit of rows may extend in the direction of the arm.

In another alternative embodiment, a plurality of units of rows of elevations, for example four units of two or three rows of elevations each, may extend away from one another in the manner of rays. Preferably, the preferred hole with spanning webs and elevations at the edge of the hole and on the webs is located in the region in which the rays converge. Such an embodiment of the pad according to the invention can be used, for example, in the hip region or in the buttock region.

For all embodiments of the pad, the individual units may be made up of at least two rows of at least three elevations each, in the same way as the individual base unit of rows of elevations, i.e., they may, for example, comprise at least two rows of elevations, but also three rows, four rows, or more rows of elevations, and the number of elevations per row may be at least three, but also more elevations per row, for example four elevations, five elevations, six elevations, seven elevations, eight elevations, or more elevations may be provided.

In a preferred embodiment, the base element of the pad pressure element is web-shaped or plate-shaped. The preferred hole is then located in the web or in the plate. The elevations project out of the web or the plate.

In a preferred embodiment, the elevations are stud-shaped or knob-shaped. In a preferred embodiment, the elevations of the rows are stud-shaped or knob-shaped. In a preferred embodiment, the elevations at the hole are stud-shaped or knob-shaped.

In a preferred embodiment, at least a portion of the elevations is embedded in the pad base body. Preferably, the elevations are completely embedded in the pad base body. Particularly preferably, the entire pad pressure element, i.e., the elevations and the base element, is embedded in the pad base body, and the pad pressure element is therefore preferably completely enclosed by the pad base body, for example molded into the pad base body.

The preferred complete sheathing of the at least one pad pressure element with the material of the pad base body not only results in the described application advantage, but also advantageously results in a simpler possibility of manufacturing the pad according to the invention, since the second base of the pad base body, which faces away from the user, can thus be readily manufactured so as to be flat.

A pad comprising a pad base body consisting of a material having a first hardness and at least one pad pressure element according to the invention consisting of a material having a second hardness is preferred, the pad base body comprising a first base facing the user and a second base facing away from the user, the surface of the at least one pad pressure element being completely surrounded by the material of the pad base body.

The pad pressure element is preferably a pressure amplification element. A pressure amplification element is used to provide a more pronounced punctiform pressure than the areal pressure exerted by the pad base body.

In a preferred embodiment, the base element and the elevations are formed in one piece, in particular they are formed from the same material.

In a preferred embodiment, the pad base body comprises a first base facing the user and a second base facing away from the user, the elevations projecting from the base element positioned in the region of the second base, which faces away from the user, and projecting in the direction of the first base facing the user.

The elevations point from the pad base body in the direction of the user, and therefore the head lies in the direction of the first base of the pad base body, and the foot and the base element lie in the direction of the second base of the pad base body.

In a preferred embodiment, the at least elevations have a height of at least 1 mm and at most 10 mm, for example approximately 3 mm. In a preferred embodiment, the elevations have a height of at least 2 mm. In a preferred embodiment, the elevations have a height of at least 3 mm. The height is measured from the base element.

The pad is preferably viscoelastic at least in part, and thus preferably has at least one viscoelastic portion. In particular, the pad is preferably viscoelastic at least in the region of the pad pressure element.

The pad base body preferably comprises a viscoelastic material at least in the region of the pad pressure element. Preferably, the pad base body is viscoelastic at least in one portion. The pad base body is preferably viscoelastic. The pad base body preferably consists of a viscoelastic material.

The material of the pad base body is preferably plastics material, silicone, or rubber. However, other suitable materials for a pad base body are also known to a person skilled in the art from the prior art.

For example, the material may be thermoplastic elastomers. Polyurethanes, for example, are also suitable.

Preferably, the material of the pad base body is bendable and/or stretchable, such that the pad base body can adapt to the body shape of the user when the pad is applied.

The pad base body material is preferably not a textile material. In an alternative embodiment, however, the pad base body may also be surrounded or otherwise covered by a textile sheath.

The pad base body material may, of course, also consist of mixtures of materials, in particular at least two of the above-mentioned materials.

The material of the pad pressure element is preferably a flexible material, in particular a flexible plastics material. In a preferred embodiment, the material of the at least one pad pressure element is plastics material, silicone, or rubber. Of course, the material of the at least one pad pressure element may also consist of mixtures of materials, in particular at least two of the above-mentioned materials.

Therefore, the stud/base element structure according to the invention can advantageously be manufactured easily, for example by means of injection molding technology.

According to the invention, the first material of the pad base body is softer than the second material of the pad pressure element.

In a preferred embodiment, the material of the pad base body has a Shore hardness of at least 10 Shore OO and at most 50 Shore OO.

The material of the pad base body, i.e., the first material, preferably has a hardness of at most 45 Shore OO, preferably at most 40 Shore OO, particularly preferably at most 30 Shore OO. The material of the pad base body, i.e., the first material, preferably has a hardness of at most 25 Shore OO, preferably at most 20 Shore OO, particularly preferably at most 19 Shore OO.

The material of the pad base body preferably has a hardness of at least 10 Shore OO, in particular at least 14 Shore OO, particularly preferably at least 15 Shore OO.

In a preferred embodiment, the material of the pad pressure element has a Shore hardness of at least 10 Shore A, preferably at least 14 Shore A, and at most 80 Shore A. In a preferred embodiment, the material of the pad pressure element has a Shore hardness of at least 20 Shore A and at most 80 Shore A.

In a preferred embodiment, the material of the at least one pad pressure element has a hardness of at least 20 Shore A and at most 60 Shore A.

In a preferred embodiment, the material of the at least one pad pressure element has a hardness of at least 20 Shore A, more preferably at least 25 Shore A.

In a preferred embodiment, the material of the at least one pad pressure element has a hardness of at most 50 Shore A. In a preferred embodiment, the material of the at least one pad pressure element has a hardness of at most 49 Shore A, more preferably at most 45 Shore A.

The material of the at least one pad pressure element preferably has a hardness of from at least 35 to at most 45 Shore A. The material of the at least one pad pressure element preferably has a hardness of approximately 40 Shore A.

In a preferred embodiment, the material of the pad base body has a Shore hardness of at least 10 Shore OO and at most 50 Shore OO and/or the material of the pad pressure element has a Shore hardness of at least 10 Shore A, preferably at least 14 Shore A, and at most 80 Shore A.

In a preferred embodiment, the pad is an elbow pad, a shoulder pad, a knee pad, an ankle pad, a back pad, a pad for the arm region, or a pad for the abdominal region.

In a preferred embodiment, the pad is an elbow pad or a shoulder pad. In a preferred embodiment, the pad is a pad for a limb, in particular an arm pad. In a preferred embodiment, the pad is an elbow pad, a shoulder pad, a knee pad, an ankle pad, or a pad for the arm region. In an alternative embodiment, the pad is a back pad or a pad for the abdominal region.

In an alternative embodiment, the pad is a shoulder pad. In another alternative embodiment, the pad is a pad for the hip region or for the buttock region.

In a preferred embodiment, the pad is an elbow pad, a shoulder pad, a hip pad, or a pad for the buttock region.

The pad is preferably not a foot pad and/or a shoe sole. The pad is preferably not a shoe sole. The pad according to the invention is preferably not a shoe insert or foot insole.

In a preferred embodiment, the pad is not a back pad.

In a preferred embodiment, the pad is an elbow pad, the base body of the pad comprising a hole for positioning the pad, i.e., a base body positioning hole.

The hole in the base body is advantageously suitable for defining the positioning of the pad on the body of the patient and for positioning the pad. For example, the hole in the base body may be positioned and dimensioned such that it encompasses the bone insertion in the elbow region and thus the positioning of the pad is fixed.

The present invention also relates to an orthosis, bandage, or compression garment comprising a pad according to the invention.

The pad is advantageously not only suitable for medical products, for example orthoses or bandages, but also for other compression garments, for example in the sports sector. The pad can also be used, for example, for sports garments, in particular sports compression garments.

Other preferred subjects of the invention will become apparent from the dependent claims and the additional independent claims.

The invention is described in more detail below with reference to the following figures, without the embodiments of the invention shown therein being limited.

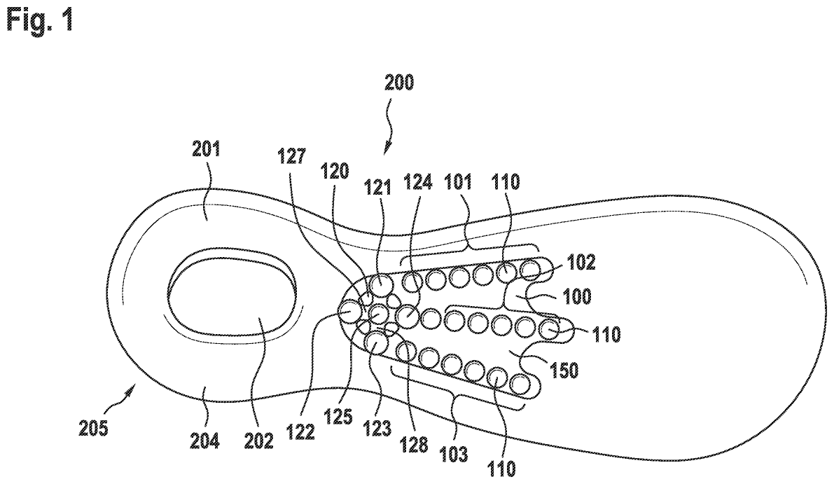

FIG. 1 shows a pad according to the invention.

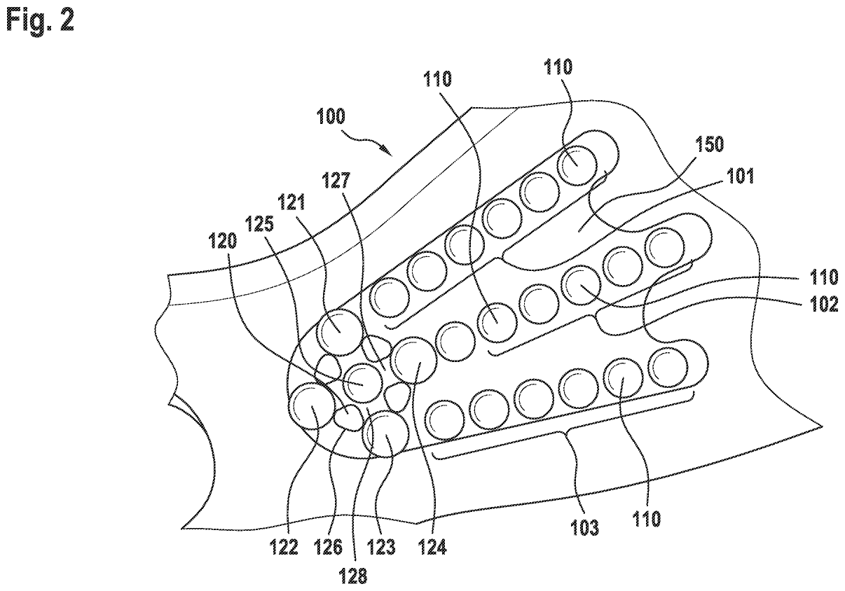

FIG. 2 shows the pad pressure element of the pad from FIG. 1.

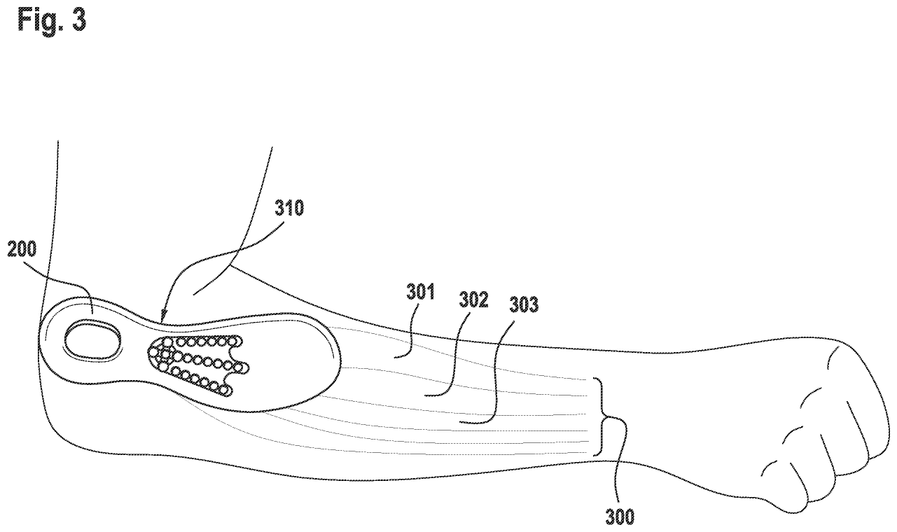

FIG. 3 shows the positioning of an elbow pad according to the invention on the arm of a person.

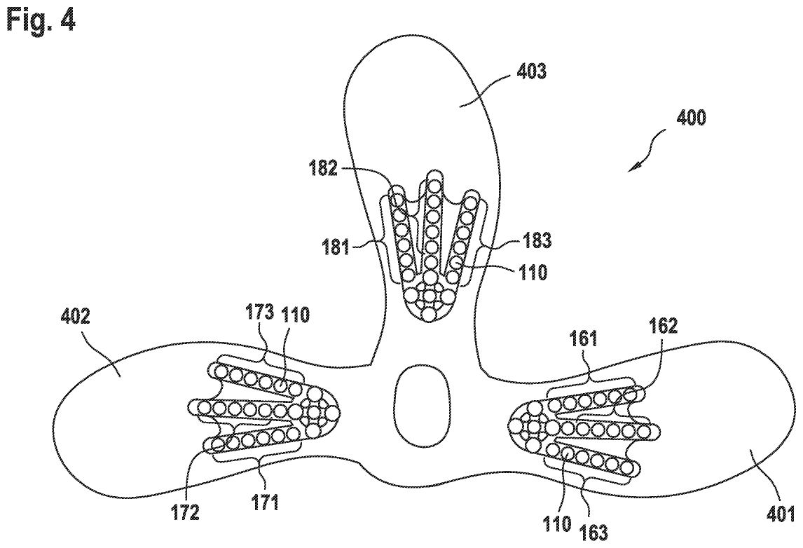

FIG. 4 shows an embodiment of the pad according to the invention comprising three units of rows of elevations.

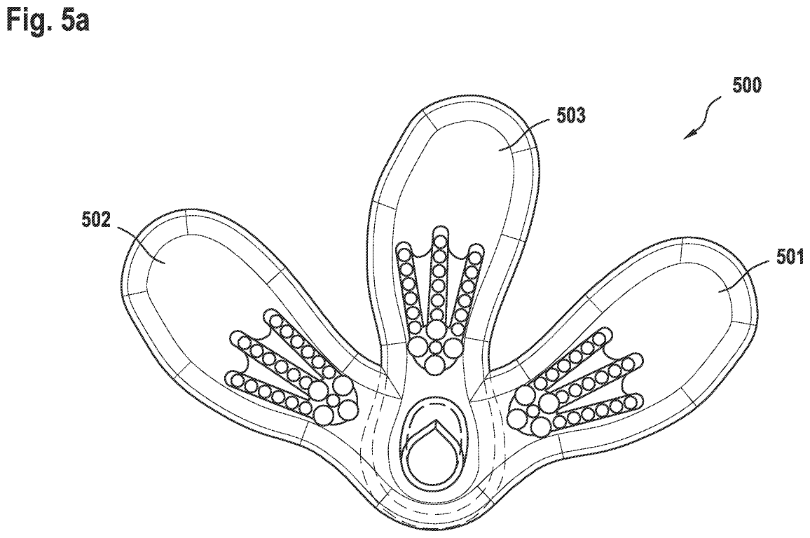

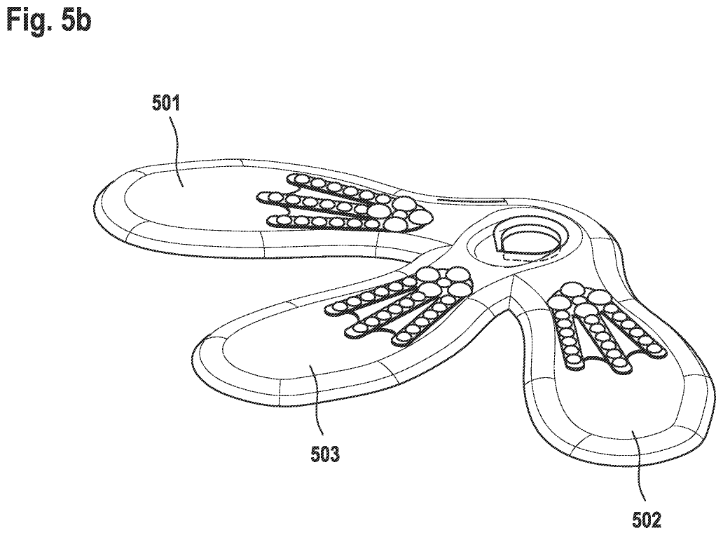

FIG. 5a shows another embodiment of the pad according to the invention comprising three units of rows of elevations.

FIG. 5b shows the embodiment from FIG. 5a in an oblique view.

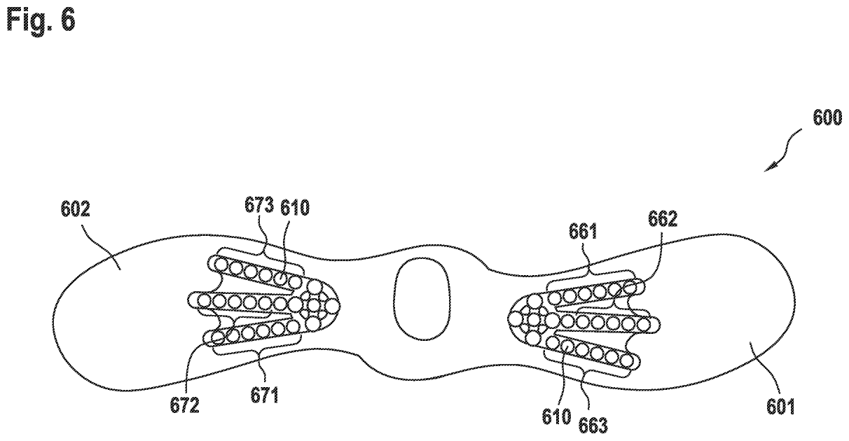

FIG. 6 shows an embodiment of the pad according to the invention comprising two units of rows of elevations.

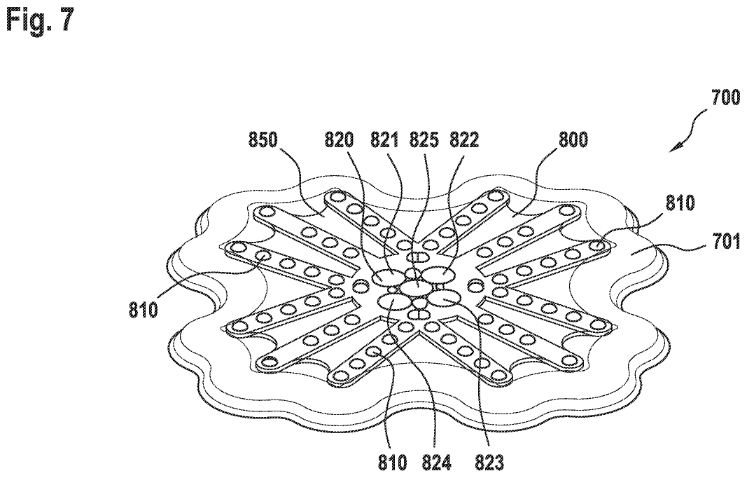

FIG. 7 shows another embodiment of the pad according to the invention comprising rows of elevations extending in the manner of rays.

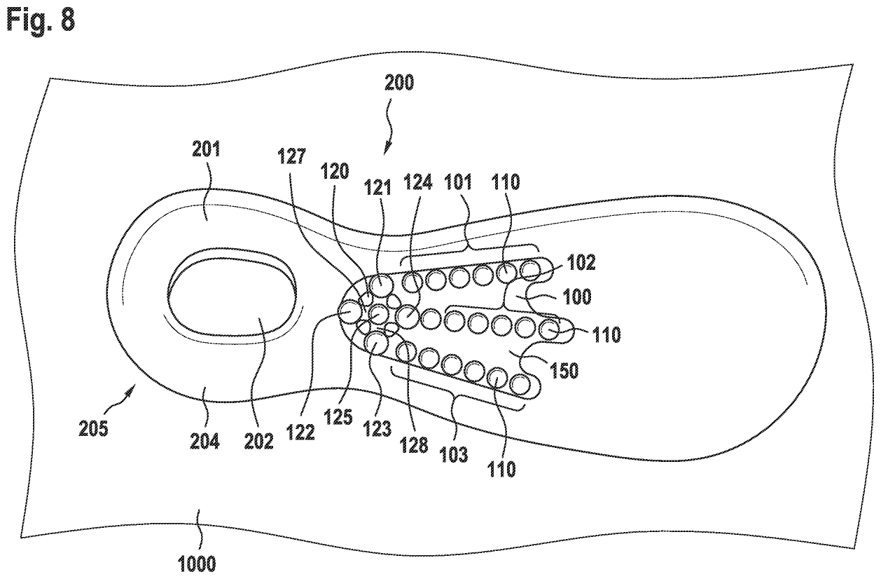

FIG. 8 shows a pad according to the invention on a knitted bandage base fabric.

FIG. 1 shows a pad (200) according to the invention, in the present case an elbow pad. The pad (200) consists of a softer pad base body (201) and a harder pad pressure element (100) embedded in the pad base body (201). The pad base body (201) comprises an annular region which forms a hole (202), which can advantageously be used for positioning the pad (200) at an elbow as shown in FIG. 3. The pad (200) comprises a front face (204) facing the body of the wearer and a rear face (205) facing away from the body of the wearer. The pad pressure element (100) is embedded completely in a tongue-shaped portion of the pad base body (201), specifically in such a way that it knob-shaped elevations (110) point from the base element (150) of the pad pressure element (100) in the direction of the front face (204) of the pad (200), i.e., in the direction of the arm of the wearer.

The pad pressure element (100) of the pad (200) from FIG. 1, which is shown by itself in FIG. 2, consists of a plate-shaped base element (150) with knob-shaped elevations (110) protruding therefrom. A first row (101), a second row (102), and a third row (103) of elevations (110) are formed on the base element (150), said elevations (110) extending in the manner of rays, i.e., not in parallel with one another. A hole (120) is located in the base element (150) at the end region of these rows (101, 102, 103). Four more elevations (121, 122, 123, 124) are positioned at the edge (126) of the hole (120). The hole is spanned by two webs (127, 128), in the point of intersection of which there is another elevation (125). On account of the hole (120), the elevations (121, 122, 123, 124, 125) located at the hole (120) are more movable than the elevation (110) arranged in the rows (101, 102, 103) on the base element (150).

FIG. 3 shows the elbow pad (200) according to the invention from FIG. 1 when worn on the arm. The tongue-shaped pad extends along the forearm muscles (300). In this case, the three rows of elevations each follow the course of different muscle fibers (301, 302, 303), such that the elevations can exert a pressure thereon over a longer course of the muscle fibers (301, 302, 303). The hole in the pad pressure element with the intersecting webs and with the elevations located at the hole and on the webs is located in the region of the tendon-bone insertion (310) of the muscle (300), such that the elevations located at the hole press on the tendon-bone insertion (310). As a result of the hole and the webs, these elevations are substantially more freely and movably resilient compared to the elevations of the rows. Therefore, on account of the pad, a different, in particular stronger, pressure is exerted on the muscle fibers (301, 302, 303) by the pad elevations there than that exerted on the tendon-bone insertion (310) by the more movable elevations in the region of the hole. The two different types of pressure in the two different regions result in a synergistic effect of the two types of elevations. The hole of the pad base body can advantageously surround the head of the humerus and thus ensure accurate and secure positioning of the pad (200).

FIG. 4 shows an elbow pad (400) according to the invention for use in the shoulder region. The pad base body consists of three tongues (401, 402, 403), each of which has a pad pressure element comprising three rows (161, 162, 163, 171, 172, 173, 181, 182, 183) of elevations (110) each. The pad (400) can encompass the upper head of the humerus by means of the hole in the pad base body, such that the rows (161, 162, 163) of the first tongue (401) are located in the region of the chest, the rows (181, 182, 183) of the third tongue (403) are located in the region of the upper arm, and the rows (171, 172, 173) of the second tongue (402) are located in the region of the back, such that a pressure can be generated on muscle fibers in all three regions.

FIG. 5a shows an alternative embodiment (500) of the shoulder pad from FIG. 4, the three tongues (501, 502, 503) being at a different angle to one another. The function of the pad from FIG. 5a is the same as that of the pad (400) from FIG. 4.

FIG. 5b shows the pad from FIG. 5a comprising the three tongues (501, 502, 503) in an oblique view.

FIG. 6 shows a pad (600) according to the invention, for example an arm pad, comprising two tongues (601, 602). The first tongue (601), with its three rows (661, 662, 663) of elevations (610), may extend along a muscle of the forearm, the second tongue (602), with its three rows (671, 672, 673) of elevations (610), may extend along a muscle of the upper arm.

FIG. 7 shows an embodiment of the pad (700) according to the invention for the hip region or the buttock region. The pad (700) comprises a star-shaped pad base body (701) in which a harder pressure element (800) having a star-shaped base element (850) is embedded. The pressure element (800) comprises a total of twelve rows of elevations (810) that diverge in the manner of rays from the center of the pad (700). In each case, three rows are combined as a unit. A hole (820) comprising two webs is located in the center of the pad pressure element (800). There are four, more movable elevations (821, 822, 823, 824) at the edge of the hole (820) and one other more movable elevation in the center of the hole (825).

FIG. 8 shows the pad (200) according to the invention from FIG. 1, which is assigned to a knitted bandage fabric (1000), for example a tubular arm bandage. The pad (200) is attached to the knitted bandage fabric (1000) by the base (205) facing away from the user.

The exact description of the pad (200) with the corresponding reference signs can be found in the description of FIG. 1.

1. A pad for orthopedic aids or compression garments, comprising:
a pad base body of a first material and at least one pad pressure element of a second material, the pad pressure element embedded in the pad base body at least in part, and the first material of the pad base body being softer than the second material of the pad pressure element; and
at least two rows of at least three elevations each, the at least two rows of elevations not extending in parallel at least in one portion, and the elevations protruding from a base element of the pad pressure element.

2. The pad according to claim 1, wherein the pad pressure element comprises three to six rows of at least three elevations (110, 610, 810) each.

3. The pad according to claim 1, wherein the rows each have four to twelve elevations.

4. The pad according to claim 1, wherein two adjacent rows of the at least two rows of at least three elevations each in the non-parallel portion extend at an angle of at least 3° and at most 25° to one another.

5. The pad according to claim 1, wherein the at least two rows of at least three elevations each in the pad base body are positioned to be located in a region of muscle strands when the pad is applied and function to exert pressure on the muscle strands.

6. The pad according to claim 1, wherein the base element of the pad pressure element comprises at least one hole, and at least two elevations being positioned on the base element at an edge of the at least one hole.

7. The pad according to claim 6, wherein the at least two elevations are connected to one another via the web spanning the hole.

8. The pad according to claim 6, wherein at least two pairs of elevations are positioned at the edge of the hole and are each connected to one another via a web spanning the hole, the webs of the two pairs of elevations intersecting, a center of the hole.

9. The pad according to claim 8, wherein another elevation is located on and/or next to a point of intersection of the two webs.

10. The pad according to claim 6, wherein the elevations positioned at the edge of the hole are assigned to the at least two rows of at least three elevations each.

11. The pad according to claim 10, wherein the hole is positioned in an end region of the at least two rows of at least three elevations each.

12. The pad according to claim 6, wherein the hole in the pad base body is positioned such that the elevations positioned at the edge of the hole are locatable in a region of a tendon-bone insertion when the pad is applied and function to exert pressure on the tendon-bone insertion.

13. The pad according to claim 1, wherein the pad pressure element comprises at least two additional rows of at least three elevations each, the at least two additional rows of elevations extending so as to be offset in an angular range of from 75° to 105° and/or so as to be offset in an angular range of from 165° to 205° with respect to the first at least two rows of elevations.

14. The pad according to claim 1, wherein the pad pressure element comprises at least two additional rows of at least three elevations each, the at least two additional rows of elevations extending so as to be offset by approximately 180° with respect to the first at least two rows of elevations.

15. The pad according to claim 1, wherein the pad pressure element comprises at least four additional rows of at least three elevations, the at least four additional rows of elevations extending so as to be offset in an angular range of from 75° to 105° and/or so as to be offset in an angular range of from 165° to 205° with respect to the first at least two rows of elevations.

16. The pad according to claim 1, wherein the pad pressure element comprises at least eight rows, preferably twelve rows, of at least three elevations each, the at least eight rows of elevations converging in the manner of rays.

17. The pad according to claim 16, further comprising a hole is located in the pad pressure element in the center of the rows of elevations that converge in a manner of rays, at least two elevations being positioned on the base element at the edge of the hole.

18. The pad according to claim 1, wherein the base element of the pad pressure element is web-shaped or plate-shaped.

19. The pad according to claim 1, wherein the elevations are stud-shaped or knob-shaped.

20. The pad according to claim 1, wherein the pad base body comprises a first base for facing a user and a second base facing away from the user, and the elevations projecting from the base element positioned in a region of the second base, which faces away from the user, and projecting in the direction of the first base, which faces the user.

21. The pad according to claim 1, wherein the pad is selected from a group consisting of an elbow pad, a shoulder pad, a knee pad, an ankle pad, a back pad, a pad for the arm region, an a pad for an abdominal region.

22. The pad according to claim 1, wherein the pad is an elbow pad, the base body of the pad comprising a hole for positioning the pad.

23. An orthosis, bandage, or compression garment comprising a pad according to claim 1.
